(19) United States
(12) Patent Application Publication
(10) Pub. No.: US 2007/0048761 A1
(43) Pub. Date: Mar. 1, 2007

(54) System and method for authenticating multiple components associated with a particular product

(75) Inventors: Paul Reep, Marina del Rey, CA (US); Ming-Hwa Liang, Stony Brook, NY (US); Sheu Jun-Jei, Stony Brook, NY (US)

Correspondence Address: VIRTUAL LEGAL, P.C.;MICHAEL A. KERR, 3476 EXECUTIVE POINTE WAY, UNIT 16, CARSON CITY, NV, 89706, US

(73) Assignee: Applied DNA Sciences, Inc.

(21) Appl. No.: 11/437,265

(22) Filed: May 19, 2006

Related U.S. Application Data
(60) Provisional application No. 60/682,976, filed on May 20, 2005.

(51) Int. Cl. C12Q 1/68

(52) U.S. Cl. 435/6

(57) ABSTRACT

A method for authenticating a product where the product is composed of a plurality of components is described. The method for authenticating the product comprises providing a liquid compound, e.g. paint, that comprises at least one invisible market, e.g. nucleic acid, in which the compound is associated with a first entity. The method then proceeds to apply the compound to the surface of the product at the juncture of at least two components. The method also comprises associating the application of the compound to the at least two components with the first entity.

### CROSS-REFERENCE

This application is related to U.S. Provisional Application No. 60/682,976 having a filing date of May 20, 2005, which is entitled “SYSTEM AND METHOD FOR AUTHENTICATING MULTIPLE COMPONENTS ASSOCIATED WITH A PARTICULAR PRODUCT,” and which is incorporated herein by reference.

### BACKGROUND

1. Field of the Invention

This invention relates to a system and method for marking a particular product having multiple components. More particularly, the invention is related to a system and method for marking products to authenticate that a particular product is genuine.

2. Description of Related Art

With the dawn of the information age comes the ability to duplicate, change, alter and distribute just about anything. The FBI has called counterfeiting the crime of the 21^{st} century. Product counterfeiting is a serious and growing threat. Measures to defend against counterfeiters are being taken by many corporations, but they have not developed comprehensive, systematic, and cost-effective solutions to preventing counterfeiting.

Due to advancing counterfeiting techniques, traditional anti-counterfeit technologies are becoming obsolete. Additionally, governments and corporations that have invested a great deal of resources in fighting counterfeiting have experienced little success. Furthermore, law enforcement agencies that are burdened with efforts to combat violent crimes have insufficient resources to fight the “victimless” counterfeiting crime.

Counterfeiting is extending to specific products that are incorporated into much more complex integrated products, and may adversely impact the effectiveness of the integrated product. Thus, a simple counterfeit fastener, which appears to be similar to a much strong fastener, may be unintentionally integrated into a complex integrated product such as an airplane wing. When the airplane wing experiences some type of material failure, the frail counterfeit fastener may be the cause of this failure.

One way to avoid counterfeiting is to authenticate products during manufacturing or during the maintenance of the integrated product. For example, fasteners are examined visually by applying a “torque stripe” to fasteners. A torque stripe may be used to determine if a fastener needs to be tightened. A torque stripe is a colored compound that is used to determine if the fastener has been loosened. For example, if the fastener is a nut and a bolt, the torque stripe is a mark that is put on the nut and bolt after it is tightened. If that fastener has come loose, an inspection will reflect that the torque stripe has separated.

A torque stripe is also used for electrical/electronic connectors that use backshells or camlocks, which seat a connection. If someone tampers with or removes and replaces the connector, the torque stripe will break.

### SUMMARY

A system and method for authenticating a product wherein said integrated product is composed of a plurality of components is described. The method for authenticating comprises, firstly, providing a compound having at least one invisible marker mixed therein. The compound is then associated with a particular entity (e.g. a manufacturer of aircraft fasteners). The particular entity proceeds to apply the compound to the product at the juncture of at least two components of the product. The product may then enter the supply chain, be sold directly to customers or be sold between customers. An independent party can then inspect the compound to authenticate that the product is correctly associated with the particular entity that joined the two components.

The product may be a manufactured product, a unique product, or a finished product. An illustrative manufactured product is a spare part that may be used to replace a worn part in a finished product. An illustrative unique product is a painting housed within a frame. An illustrative finished product is a metal fastener used to assemble an aircraft.

The compound may be a paint, ink, paste, emulsion, glue, adhesive, or other such compound that may be mixed and/or integrated with an invisible marker. In the illustrative embodiment, the compound is paint, which is combined with the invisible marker or taggant. The marker is selected from a taggant group that includes but is not limited to a nucleic acid taggant, a DNA taggant, a luminescent taggant(s), a phosphorescent taggant(s), a chemiluminescent taggant(s), a fluoroluminescent taggant(s), an optical or machine readable taggant, a nano-particle taggant, a micro-sphere taggant, a probe insertion for surrogate authentication of the DNA, a chemical taggant having a visible, infra-red, near infra-red and ultra-Violet absorber and reflector component chemistry, a taggant that is reusable, a color-shifting ink taggant, a pigment taggant, a catalyst taggant, a taggant that has an antigenic reaction for instant, non-forensic assay, with swab swipe stylus. In one illustrative embodiment, the taggant is an invisible marker such as a nucleic acid, or UV ink.

In the illustrative embodiment, the present disclosure describes the use of a marker into a torque stripe compound/material that may be used to verify claims that the assembly is a factory original and unaltered and has not been damaged or replaced, verify that the date of assembly can be confirmed, verify factory origin, or speed up QA/QC operations.

### BRIEF DESCRIPTION OF THE DRAWINGS

FIG. 1 is a flow chart of one embodiment of the methods of the invention.

FIG. 2 is a flow chart of one embodiment of the method of authenticating a fastener with a torque stripe, the torque stripe comprising a DNA taggant in accordance with the invention.

FIG. 3 is a graphical representation of real time PCR results illustrating the detection of a DNA taggant from a torque stripe in accordance with one embodiment of the methods of the invention.

FIG. 4 is a graphical representation of real time PCR results illustrating the detection of a DNA taggant from a torque stripe in accordance with yet another embodiment of the methods of the invention.

### DESCRIPTION

Before the present methods for authenticating products are described, it is to be understood that this invention is not limited to the particular product described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a taggant” includes a plurality of such taggants and reference to “the primer” includes reference to one or more primers and equivalents thereof known to those skilled in the art, and so forth.

The publications discussed herein are provided solely for their disclosure prior to the filing date of the present application. Nothing herein is to he construed as an admission that the present invention is not entitled to antedate such publication by virtue of prior invention. Further, the dates of publication provided may be different from the actual publication dates which may need to be independently confirmed.

Although the description about the methods for authenticating a particular product contains many limitations in the specification, these should not be construed as limiting the scope of the claims but as merely providing illustrations of some of the presently preferred embodiments of this invention. Many other embodiments will be apparent to those of skill in the art upon reviewing the description. Thus, the scope of the invention should be determined by the appended claims, along with the full scope of equivalents to which such claims are entitled.

A “Nucleic acid tag” is a nucleic acid oligomer or fragment used to identify or authenticate a particular product. Nucleic acid tag and nucleic acid taggant are interchangeable throughout the specification.

The term “DNA taggant” means a nucleic acid tag which comprises deoxy nucleotides. A DNA taggant maybe double stranded or single stranded, cDNA, STR (short tandem repeats) and the like. The DNA taggant may also comprise modification to one or more nucleotides which aid in the identification or detection of the DNA taggant.

The term “DNA marker compound” means a marker compound utilized to identify or authenticate a particular product which comprises a specific DNA oligomer which is used to authenticate the particular product.

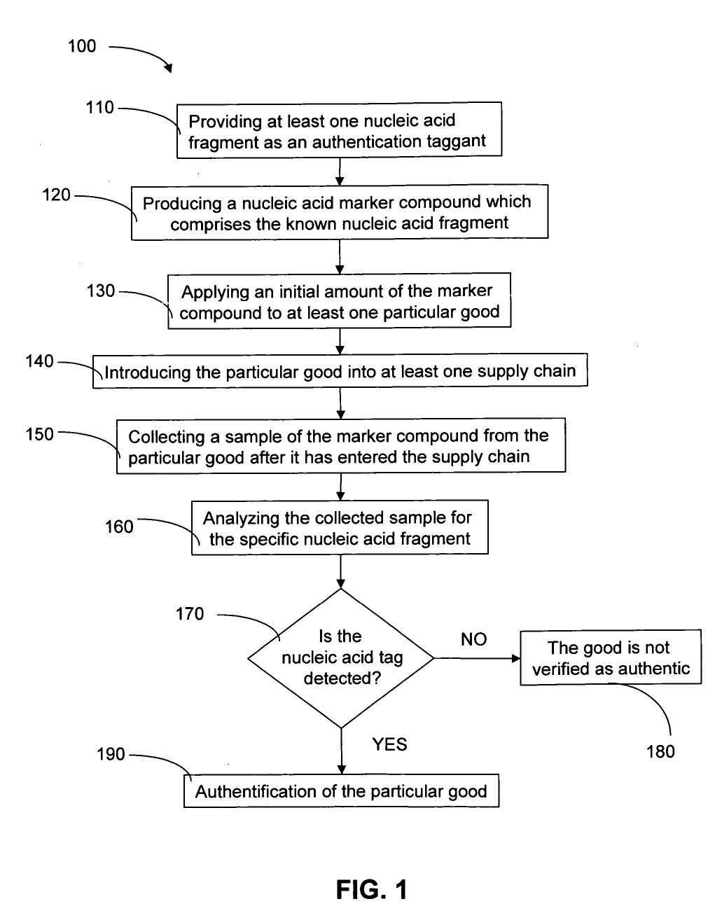

A method for labeling an object or product with a specified nucleic acid tag and then detecting the nucleic acid tag in the object or product in an effective manner is described. FIG. 1 shows a flow chart of the general process 100 of introducing a nucleic acid tag into or onto a product and being able to detect the nucleic acid tag or marker incorporated in the product. The process comprises providing at least one specific nucleic acid fragment as an authentication tag or marker for a product in event 110. The nucleic acid marker maybe DNA, cDNA, or any other nucleic acid fragment comprising nucleic acids or nucleic acid derivatives. The marker may be an nucleic acid fragment that is single stranded or preferably, double stranded and may vary in length, depending on the product to be labeled as well as the detection technique utilized in the nucleic acid marker detection process.

The nucleic acid marker may be synthetically produced using a nucleic acid synthesizer or by isolating nucleic acid material from yeast, human cell lines, bacteria, animals, plants and the like. In certain embodiments, the nucleic acid material may be treated with restriction enzymes and then purified to produce an acceptable nucleic acid marker(s). The length of the nucleic acid marker/tag usually ranges between about 100 bases to about 10 kilo bases, more usually about 500 bases to about 6 kb, and preferably about 1 kb to about 3 kb in length.

The nucleic acid taggant may comprise one specific nucleic acid sequence or alternatively, may comprise a plurality of various nucleic acid sequences. In one embodiment, polymorphic DNA fragments of the type short tandem repeats (STR) or single nucleotide polymorphisms (SNP) are utilized as an anti-counterfeit nucleic acid tag. While the use of a single sequence for a nucleic acid marker may make detection of the marker easier and quicker, the use of a plurality of nucleic acid sequences such as STR and SNP, in general, give a higher degree of security against forgers.

In certain embodiments of the methods of the invention, the nucleic acid marker is derived from DNA extracted from a specific plant source and is specifically digested and ligated to generate artificial nucleic acid sequences which are unique to the world. The digestion and ligation of the extracted DNA is completed by standard restriction digestion and ligation techniques known to those skilled in the art of molecular biology. Once the modified DNA taggant has been produced, the taggant is encapsulated into materials for protection against UV and degradation. The DNA encapsulant materials are generally of plant origin.

After the nucleic acid fragment with a known nucleic acid sequence has been manufactured or isolated, the method further comprises producing a DNA marker compound which comprises the selected nucleic acid fragment in event 120. The marker compound maybe produced as a solid or liquid, water or oil based, a suspension, an aggregate and the like. An important feature of the marker compound is to protect the nucleic acid fragment from UV and other degradation factors that may degrade the nucleic acid taggant overtime, while the nucleic acid is acting as an authentication tag for a particular product. In certain embodiments, when the taggant is DNA, the nucleic acid tag may be encapsulated and suspended in a solvent solution (aqueous or organic solvent solution) producing a “stock” DNA taggant solution at a specified concentration. This stock DNA solution can then be easily be added to the marker compound mixture at an appropriate concentration for the type of product to be authenticated. In certain instances, the DNA taggant may be mixed with other components of the marker compound without any prior encapsulation. Processes such as nucleic acid fragment encapsulation and other techniques utilized for protecting nucleotides, and in particular, DNA from degradation are described more fully below.

Another important feature of the marker compound mixture is to be able to camouflage or “hide” the specified nucleic acid tag with extraneous and nonspecific nucleic acid oligomers/fragments, thus making it difficult for unauthorized individuals, such as forgers to identify the sequence of the nucleic acid tag. In certain embodiments, the marker compound comprises a specified dsDNA taggant from a known source (i.e. mammal, invertebrate, plant and the like) along with genomic DNA from the corresponding or similar DNA source. The amount of the DNA taggant found in a marker compound varies depending on the particular product to be authenticated, the duration the taggant needs to be viable (e.g. 1 day, 1 month, 1 year, multiple years) prior to authentication, expected environmental exposure, the detection method to be utilized, etc.

In general, when the taggant is dsDNA, and PCR is the technique for taggant detection. The copy number of DNA taggant in a predetermined sample size of marker compound used for authentification is about 3 copies to about 100,000 copies, more usually about 10 copies to about 50,000 copies, and even more usually about 100 copies to about 10,000 copies of DNA taggant.

After the nucleic acid fragment with a known nucleic acid sequence has been manufactured or isolated, and added to a marker compound mixture, the process for authenticating a particular product by detecting a nucleic acid tag, further comprises applying a predetermined amount of nucleic acid marker to a specified product in event 130. The particular product may be tagged with a nucleic acid marker throughout the complete product or only in a predetermined region of the product. When the product to be authenticated is a solid, a specified amount of nucleic acid marker may be incorporated throughout the volume of the product, only on the surface of the product or in some embodiments, placed only on a previously designated section of the product. When the product is a prescription drug, either in solid or liquid form, the drug (i.e. pills, gel capsules, etc.) could have the nucleic acid tag incorporated completely throughout the product. If the product is a prescription drug in tablet form, the nucleic acid marker compound may be in the form of a solid which can be introduced into the product (drug) during the compression of the drug into a tablet. If the product is a textile garment, the marker could be either solid or liquid and applied to a predetermined area of the garment. Textiles may have a label with the manufactures name on it and may also be used as a region of the product which the nucleic acid marker is placed. The above examples are presented for clarity and are not meant to be limiting in scope.

The embodiment of the method of authenticating a product depicted in FIG. 1 further comprises introducing the marked product into a supply chain or placing the product into service in event 140. Frequently, forgers have the best access to products when they are being shipped from the manufacturer/producer to a retail outlet or location. Forgers also have access to the products of interest during maintenance or service of certain of products, such as aircraft, where the product of interest is inspected or replaced (i.e. fasteners). Having a method in which the producer can track and authenticate its products allows for a better monitoring of when and where products are being replaced with forgeries or being tampered with.

In event 150, a sample is collected from the particular product comprising the nucleotide tag after it has entered the supply chain or been in service. A manufacturer or an authorized individual can collect a sample of the marker compound from the product at any desired point along the supply chain or during the service or routine maintenance of an item where the product is utilized for authentication purposes. In certain embodiments, this may comprise visually inspecting the marker compound, and/or scraping, cutting or dissolving a portion of the marker compound in a solvent for analysis.

The embodiment shown in FIG. 1 further comprises analyzing the collected sample for the presence of the nucleic acid taggant in event 160. The analysis of the sample collected from the product may occur without further purification, but usually, some extraction, isolation or purification of the nucleic acid tag obtained in the sample is required. Details on the extraction, concentration and purification techniques useful for the methods of the invention are described more fully below and also in the examples.

In general, analyzing the sample comprises providing a “detection molecule” configured to the nucleic acid tag. A detection molecule includes but is not limited to a nucleic acid probe and/or primer set which is complementary to the sequence of the nucleic acid taggant, or a dye label or color producing molecule configured to bind and adhere to the nucleic acid taggant. When the detection of the nucleic acid taggant comprises amplifying the nucleic acid taggant using PCR, the detection molecule(s) are primers which specifically bind to a certain sequence of the nucleic acid taggant. When real time PCR is utilized in the analysis of the sample, an identifiable nucleotide probe may also be provided to enhance the detection of the nucleic acid taggant as well as provide semi-quantitative or quantitative authentication results. With the use of real time PCR, results from the analysis of the sample can be completed within 30 minutes to 2 hours, including extracting or purifying the nucleic acid taggant from the collected sample. Various embodiments utilize a wide range of detection methods besides for PCR and real time PCR, such as fluorescent probes, probes configured to molecules which allow for the detection of the nucleic acid tag when bound to the probe by Raman spectroscopy, Infrared spectroscopy or other spectroscopic techniques used by those skilled in the art of nucleic acid detection.

In event 170 the results of the analysis of the collected sample are reviewed to determine if the specific nucleic acid taggant was detected in the sample. If the nucleic acid taggant is not found or detected in the collected sample of the product of interest, the conclusion from the analysis is that the product is not authentic or has been tampered with at event 180 of FIG. 1. If the nucleic acid taggant is detected in the sample at event 190, then the product is verified as being authentic.

In some embodiments, the quantity or concentration of the nucleic acid taggant within a collected sample can be determined and compared to the initial amount of nucleic acid taggant placed in the product to allow for the detection of fraud caused by diluting the product with inferior products by forgers. In general, quantitative detection methods comprise providing an internal or external control to evaluate the efficiency of detection from one sample/analysis to the next. The efficiency of detection may be affected by many parameters such as, probe hybridization conditions, molecules or substances in the product which may interfere with detection, and/or primer integrity, enzyme quality, temperature variations for detection methods utilizing PCR. By providing a control, in the detection methods, any variable conditions can be normalized to obtain an accurate final concentration of the nucleic acid tag in the product.

Incorporation of Functional Groups

In certain embodiments, the nucleic acid tag is labeled with at least one compound or “detection molecule” prior to being incorporated into the specified product to aid in the extraction and/or detection of the nucleic acid marker from the product after being placed in a supply chain. A detection molecule is a molecule or compound with at least one functionality. For example, fluorescent molecules may be configured to the nucleic acid marker for certain detection methods which are described in detail below.

In certain preferred aspects, suitable dyes include, but are not limited to, coumarin dyes, xanthene dyes, resorufins, cyanine dyes, difluoroboradiazaindacene dyes (BODIPY), ALEXA dyes, indoles, bimanes, isoindoles, dansyl dyes, naphthalimides, phthalimides, xanthenes, lanthanide dyes, rhodamines and fluoresceins. In other embodiments, certain visible and near IR dyes are known to be sufficiently fluorescent and photostable to be detected as single molecules. In this aspect the visible dye, BODIPY R6G (525/545), and a larger dye, LI-COR's near-infrared dye, IRD-38 (780/810) can be detected with single-molecule sensitivity and are used to practice the authentication process described herein. In certain embodiments, suitable dyes include, but are not limited to, fluorescein, 5-carboxyfluorescein (FAM), rhodamine, 5-(2′-aminoethyl) aminonapthalene-1-sulfonic acid (EDANS), anthranilamide, coumarin, terbium chelate derivatives, Reactive Red 4, BODIPY dyes and cyanine dyes.

There are many linking moieties and methodologies for attaching fluorophore or visible dye moieties to nucleotides, as exemplified by the following references: Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19: 3019 (1991) (3′ sulfhydryl); Giusti et al., PCR Methods and Applications, 2: 223-227 (1993) and Fung et al., U.S. Pat. No. 4,757,141 (5′ phosphoamino group via Aminolink™ II available from Applied Biosystems, Foster City, Calif.) Stabinsky, U.S. Pat. No. 4,739,044 (3′ aminoalkylphosphoryl group); AP3 Labeling Technology (U.S. Pat. Nos. 5,047,519 and 5,151,507, assigned to E.I. DuPont de Nemours & Co); Agrawal et al, Tetrahedron Letters, 31: 1543-1546 (1990) (attachment via phosphoramidate linkages); Sproat et al., Nucleic Acids Research, 15: 4837 (1987) (5′ mercapto group); Nelson et al, Nucleic Acids Research, 17: 7187-7194 (1989) (3′ amino group); and the like.

In other embodiments, the complementary nucleic acid probe is labeled with at least one compound or molecule with functionality to aid in the detection of the nucleic acid tag/marker. The techniques and dyes utilized in labeling the nucleic acid tag or the complementary probe are the same due to the nucleic acid nature of the tag and probe.

The detection molecules of the invention can be incorporated into probe motifs, such as Taqman probes (Held et al., Genome Res. 6: 986-994 (1996), Holland et al., Proc. Nat. Acad. Sci. USA 88: 7276-7280 (1991), Lee et al., Nucleic Acids Res. 21: 3761-3766 (1993)), molecular beacons; Tyagi et al., Nature Biotechnol., 16:49-53 (1998), U.S. Pat. No. 5,989,823, issued Nov. 23, 1999)) scorpion probes (Whitcomb et al., Nature Biotechnology 17: 804-807 (1999)), sunrise probes (Nazarenko et al., Nucleic Acids Res. 25: 2516-2521 (1997)), conformationally assisted probes (Cook, R., copending and commonly assigned U.S. Provisional Application No. 60/138,376, filed Jun. 9, 1999), peptide nucleic acid (PNA)-based light up probes (Kubista et al., WO 97/45539, December 1997), double-strand specific DNA dyes (Higuchi et al, Bio/Technology 10: 413-417 (1992), Wittwer et al, Bio/Techniques 22: 130-138 (1997)) and the like. These and other probe motifs with which the present detection molecules can be used are reviewed in Nonisotopic DNA Probe Techniques, Academic Press, Inc. 1992.

In some embodiments the molecular beacon system is utilized to detect and quantify the nucleic acid tag from the product of interest. “Molecular beacons” are hairpin-shaped nucleic acid detection probes that undergo a conformational transition when they bind to their target that enables the molecular beacons to be detected. In general, the loop portion of a molecular beacon is a probe nucleic acid sequence which is complementary to the nucleic acid marker. The stem portion of the molecular beacon is formed by the annealing of arm sequences of the molecular beacon that are present on either side of the probe sequence. A functional group such as a fluorophore (e.g. coumarin, EDNAS, fluorescein, lucifer yellow, tetramethylrhodamine, texas red and the like) is covalently attached to the end of one arm and a quencher molecule such as a nonfluorescent quencher (e.g. DABCYL) is covalently attaches to the end of the other arm. When there is no target (nucleic acid tag) present, the stem of the molecular beacon keeps the functional group quenched due to its close proximity to the quencher molecule. However, when the molecular beacon binds to their specified target, a conformational change occurs to the molecular beacon such that the stem and loop structure cannot be formed, thus increasing the distance between the functional group and the quencher which enables the presence of the target to be detected. When the functional group is a fluorophore, the binding of the molecular beacon to the nucleic acid tag is detected by fluorescence spectroscopy.

In certain embodiments a plurality of nucleic acid tags with varying sequences are used in labeling a particular product. The different nucleic acid tags can be detected quantitatively by a plurality of molecular beacons, each with a different colored lo fluorophore and with a unique probe sequence complementary to at least one of the plurality of nucleic acid tags. Being able to quantitate the various fluorphores (i.e. various nucleic acid tags) provides a higher level of authentication and security. It should be noted, that the other functional groups described above useful in labeling nucleic acid probes can also be utilized in molecular beacons for the present invention.

Encapsulation of a Nucleic Acid Tag

In some embodiments, the nucleic acid marker is incorporated into the product in the presence of molecules which encapsulate the nucleic acid marker by forming microspheres. Encapsulating the nucleic acid marker has the benefit of preventing the 20 nucleic acid marker from degrading while present in a supply chain or during the use of the marked product. The encapsulating materials in most embodiments are of plant origin but may also be synthetically produced materials. The encapsulation of a nucleic acid tag comprises placing the nucleic acid tag into a solvent with a polymer configured to form a microshpere around the tag. The polymers used can be selected from biodegradable or non-biodegradable polymers. Preferred biodegradable polymers are those such as lactic and glycolic acids and esters such as polyanhydrides, polyurethantes, butryic polyacid, valeric polyacid, and the like. Non biodegradable polymers appropriate for encapsulation are vinyletylenene acetate and acrylic polyacid, polyamides and copolymers as a mixture thereof. The polymers can also be selected from natural compounds such as dextran, cellulose, collagen, albumin, casein and the like.

Certain aspects of the invention comprise labeling the microspheres to benefit in the capture of the nucleic acid tag during the extraction of the label from the product of interest. The microspheres may comprise magnetically charged molecules which allow the microspheres containing the nucleic acid tag to be pulled out of a solution by a magnet.

The microspheres can also be labeled with streptavidin, avidin, biotinylated compounds and the like. Labeling the microspheres aids in the purification of the nucleic acid tag prior to detection and also is useful in concentrating the nucleic acid tag so as to enable in some embodiments, the nucleic acid tag to be detected without PCR amplification.

In other embodiments, the nucleic acid marker is applied or added to the product without being encapsulated in microspheres. For example, the nucleic acid marker may be dissolved in a solution compatible with the composition of the particular product such as a textile and then the solution comprising the nucleic acid marker is placed on the surface of the textile product, allowing the nucleic acid marker to be attached on the surface of the fabric or to be absorbed into the fabric.

Incorporation of the Nucleic Acid Tag into the Particular Product of Interest

The method of incorporating the nucleic acid tag into a product depends significantly on the type of product to be authenticated as described above. The nucleic acid tag maybe added to a marker compound in a “naked” or encapsulated form at a predetermine concentration which allows for accurate detection of the nucleic acid taggant. The marker compound is generally a liquid but in certain embodiments is a solid. The marker compound may be a liquid and after the addition of the nucleic acid taggant, is dried prior to introducing the marker as an inert substance of a particular product (e.g. a drug tablet, textile). When the marker compound comprising a nucleic acid taggant is in liquid form, the marker compound is generally applied to the product in a lacquer, paint or liquid aerosol form.

Nucleic Acid Tag Extraction and Capture Methods

A variety of nucleic acid extraction solutions have been developed over the years for extracting nucleic acid sequences from a sample of interest. See, for example, Sambrook et al. (Eds.) Molecular Cloning, (1989) Cold Spring Harbor Press. Many such methods typically require one or more steps of, for example, a detergent-mediated step, a proteinase treatment step, a phenol and/or chloroform extraction step, and/or an alcohol precipitation step. Some nucleic acid extraction solutions may comprise an ethylene glycol-type reagent or an ethylene glycol derivative to increase the efficiency of nucleic acid extraction while other methods only use grinding and/or boiling the sample in water. Other methods, including solvent-based systems and sonication, could also be utilized in conjunction with other extraction methods.

In some embodiments, the authentication process comprises capturing the nucleic acid tag directly with a complementary hybridization probe attached to a solid support. In general, the methods for capturing the nucleic acid tag involve a material in a solid-phase interacting with reagents in the liquid phase. In certain aspects, the nucleic acid probe is attached to the solid phase. The nucleic acid probe can be in the solid phase such as immobilized on a solid support, through any one of a variety of well-known covalent linkages or non-covalent interactions. In certain aspects, the support is comprised of insoluble materials, such as controlled pore glass, a glass plate or slide, polystyrene, acrylamide gel and activated dextran. In other aspects, the support has a rigid or semi-rigid character, and can be any shape, e.g. spherical, as in beads, rectangular, irregular particles, gels, microspheres, or substantially flat support. In some embodiments, it can be desirable to create an array of physically separate sequencing regions on the support with, for example, wells, raised regions, dimples, pins, trenches, rods, pins, inner or outer walls of cylinders, and the like. Other suitable support materials include, but are not limited to, agarose, polyacrylamide, polystyrene, polyacrylate, hydroxethylmethacrylate, polyamide, polyethylene, polyethyleneoxy, or copolymers and grafts of such. Other embodiments of solid-supports include small particles, non-porous surfaces, addressable arrays, vectors, plasmids, or polynucleotide-immobilizing media.

As used in the methods of capturing the nucleic acid tag, a nucleic acid probe can be attached to the solid support by covalent bonds, or other affinity interactions, to chemically reactive functionality on the solid-supports. The nucleic acid can be attached to solid-supports at their 3′, 5′, sugar, or nucleobase sites. In certain embodiments, the 3′ site for attachment via a linker to the support is preferred due to the many options available for stable or selectively cleavable linkers. Immobilization is preferably accomplished by a covalent linkage between the support and the nucleic acid. The linkage unit, or linker, is designed to be stable and facilitate accessibility of the immobilized nucleic acid to its sequence complement. Alternatively, non-covalent linkages such as between biotin and avidin or streptavidin are useful. Examples of other functional group linkers include ester, amide, carbamate, urea, sulfonate, ether, and thioester. A 5′ or 3′ biotinylated nucleotide can be immobilized on avidin or steptavidin bound to a support such as glass.

Depending on the initial concentration of the nucleic acid tag added to the product of interest, the tag can be detected quantitatively without being amplified by PCR. In some embodiments, a single stranded DNA tag labeled with a detection molecule (i.e. fluorophore, biotin, etc.) can be hybridized to a complementary probe attached to a solid support to allow for the specific detection of the “detection molecule” configured to the tag. The nucleic acid DNA tag can also be double stranded, with at least one strand being labeled with a detection molecule. With a dsDNA tag, the nucleic acid tag must be heated sufficiently and then quick cooled to produce single stranded DNA, where at least one of the strands configured with a detection molecule is capable of hybridizing to the complementary DNA probe under appropriate hybridization conditions.

In certain aspects of the invention, the complementary probe is labeled with a detection molecule and allowed to hybridize to a strand of the nucleic acid tag. The hybridization of the probe can be completed within the product, when the product is a textile or can be completed after the nucleic acid tag/marker has been extracted from the product, such as when the products are liquid (e.g. oil, gasoline, perfume, etc.). The direct detection methods described herein depend on having a large initial concentration of nucleic acid label embedded into the product or rigorous extraction/capture methods which concentrate the nucleic acid tag extracted from a large volume or mass of a particular product.

Real-Time PCR Amplification

In many embodiments, the authentication process comprises amplifying the nucleic tag by polymerase chain reaction. However, conventional PCR amplification is not a quantitative detection method. During amplification, primer dimers and other extraneous nucleic acids are amplified together with the nucleic acid corresponding to the analyte. These impurities must be separated, usually with gel separation techniques, from the amplified product resulting in possible losses of material. Although methods are known in which the PCR product is measured in the log phase, these methods require that each sample have equal input amounts of nucleic acid and that each sample amplifies with identical efficiency, and are therefore, not suitable for routine sample analyses. To allow an amount of PCR product to form which is sufficient for later analysis and to avoid the difficulties noted above, quantitative competitive PCR amplification uses an internal control competitor and is stopped only after the log phase of product formation has been completed.

In a further development of PCR technology, real time quantitative PCR has been applied to nucleic acid analytes or templates. In this method, PCR is used to amplify DNA in a sample in the presence of a nonextendable dual labeled fluorogenic hybridization probe. One fluorescent dye serves as a reporter and its emission spectra is quenched by the second fluorescent dye. The method uses the 5′ nuclease activity of Taq polymerase to cleave a hybridization probe during the extension phase of PCR. The nuclease degradation of the hybridization probe releases the quenching of the reporter dye resulting in an increase in peak emission from the reporter. The reactions are monitored in real time. Reverse transcriptase (RT)-real time PCR (RT-PCR) has also been described (Gibson et al., 1996). Numerous commercially thermal cyclers are available that can monitor fluorescent spectra of multiple samples continuously in the PCR reaction, therefore the accumulation of PCR product can be monitored in ‘real time’ without the risk of amplicon contamination of the laboratory. Heid, C. A.; Stevens, J.; Livak, K. L.; Williams, P. W. (1996). Real time quantitative PCR. Gen. Meth. 6: 986-994.

In some embodiments of the anti-counterfeit authentication process real time PCR detection strategies may be used; including known techniques such as intercalating dyes (ethidium bromide) and other double stranded DNA binding dyes used for detection (e.g. SYBR green, a highly sensitive fluorescent stain, FMC Bioproducts), dual fluorescent probes (Wittwer, C. et al., (1997) BioTechniques 22: 176-181) and panhandle fluorescent probes (i.e. molecular beacons; Tyagi S., and Kramer F R. (1996) Nature Biotechnology 14: 303-308). Although intercalating dyes and double stranded DNA binding dyes permit quantitation of PCR product accumulation in real time applications, they suffer from the previously mentioned lack of specificity, detecting primer dimer and any non-specific amplification product. Careful sample preparation and handling, as well as careful primer design, using known techniques must be practiced to minimize the presence of matrix and contaminant DNA and to prevent primer dimer formation. Appropriate PCR instrument analysis software and melting temperature analysis permit a means to extract specificity and may be used with these embodiments.

PCR amplification is performed in the presence of a non-primer detectable probe which specifically binds the PCR amplification product, i.e., the amplified detector DNA moiety. PCR primers are designed according to known criteria and PCR may be conducted in commercially available instruments. The probe is preferably a DNA oligonucleotide specifically designed to bind to the amplified detector molecule. The probe preferably has a 5′ reporter dye and a downstream 3′ quencher dye covalently bonded to the probe which allow fluorescent resonance energy transfer. Suitable fluorescent reporter dyes include 6-carboxy-fluorescein (FAM), tetrachloro-6-carboxy-fluorescein (TET), 2,7-dimethoxy-4,5-dichloro-6-carboxy-fluorescein (JOE) and hexachloro-6-carboxy-fluorescein (HEX). A suitable reporter dye is 6-carboxy-tetramethyl-rhodamine (TAMRA). These dyes are commercially available from Perkin-Elmer, Philadelphia, Pa. Detection of the PCR amplification product may occur at each PCR amplification cycle. At any given cycle during the PCR amplification, the amount of PCR product is proportional to the initial number of template copies. The number of template copies is detectable by fluorescence of the reporter dye. When the probe is intact, the reporter dye is in proximity to the quencher dye which suppresses the reporter fluorescence. During PCR, the DNA polymerase cleaves the probe in the 5′-3′ direction separating the reporter dye from the quencher dye increasing the fluorescence of the reporter dye which is no longer in proximity to the quencher dye. The increase in fluorescence is measured and is directly proportional to the amplification during PCR. This detection system is now commercially available as the TaqMan® PCR system from Perkin-Elmer, which allows real time PCR detection.

In an alternative embodiment, the reporter dye and quencher dye may be located on two separate probes which hybridize to the amplified PCR detector molecule in adjacent locations sufficiently close to allow the quencher dye to quench the fluorescence signal of the reporter dye. As with the detection system described above, the 5′-3′ nuclease activity of the polymerase cleaves the one dye from the probe containing it, separating the reporter dye from the quencher dye located on the adjacent probe preventing quenching of the reporter dye. As in the embodiment described above, detection of the PCR product is by measurement of the increase in fluorescence of the reporter dye.

Molecular beacons systems are frequently used with real time PCR for specifically detecting the nucleic acid template in the sample quantitatively. For instance, the Roche Light Cycler™ or other such instruments may be used for this purpose. The detection molecule configured to the molecular beacon probe may be visible under daylight or conventional lighting and/or may be fluorescent. It should also be noted that the detection molecule may be an emitter of radiation, such as a characteristic isotope.

The ability to rapidly and accurately detect and quantify biologically relevant molecules with high sensitivity is a central issue for medical technology, national security, public safety, and civilian and military medical diagnostics. Many of the currently used approaches, including enzyme linked immunosorbant assays (ELISAs) and PCR are highly sensitive. However, the need for PCR amplification makes a detection method more complex, costly and time-consuming. In certain embodiments anti-counterfeit nucleic acid tags are detected by Surface Enhanced Raman Scattering (SERS) as described in U.S. Pat. No. 6,127,120 by Graham et al. SERS is a detection method which is sensitive to relatively low target (nucleic acid) concentrations, which can preferably be carried out directly on an unamplified samples. Nucleic acid tags and/or nucleic acid probes can be labeled or modified to achieve changes in SERS of the nucleic acid tag when the probe is hybridized to the nucleic acid tag. The use of SERS for quantitatively detecting a nucleic acid provides a relatively fast method of analyzing and authenticating a particular product.

Another detection method useful in the invention is the Quencher-Tether-Ligand (QTL) system for a fluorescent biosensor described in U.S. Pat. No. 6,743,640 by Whitten et al. The QTL system provides a simple, rapid and highly-sensitive detection of biological molecules with structural specificity. QTL system provides a chemical moiety formed of a quencher (Q), a tethering element (T), and a ligand (L). The system is able to detect target biological agents in a sample by observing fluorescent changes.

The QTL system can rapidly and accurately detect and quantify target biological molecules in a sample. Suitable examples of ligands that can be used in the polymer-QTL approach include chemical ligands, hormones, antibodies, antibody fragments, oligonucleotides, antigens, polypeptides, glycolipids, proteins, protein fragments, enzymes, peptide nucleic acids and polysaccharides. Examples of quenchers for use in the QTL molecule include methyl viologen, quinones, metal complexes, fluorescent dyes, and electron accepting, electron donating and energy accepting moieties. The tethering element can be, for example, a single bond, a single divalent atom, a divalent chemical moiety, and a multivalent chemical moiety. However, these examples of the ligands, tethering elements, and quenchers that form the QTL molecule are not to be construed as limiting, as other suitable examples would be easily determined by one of skill in the art.

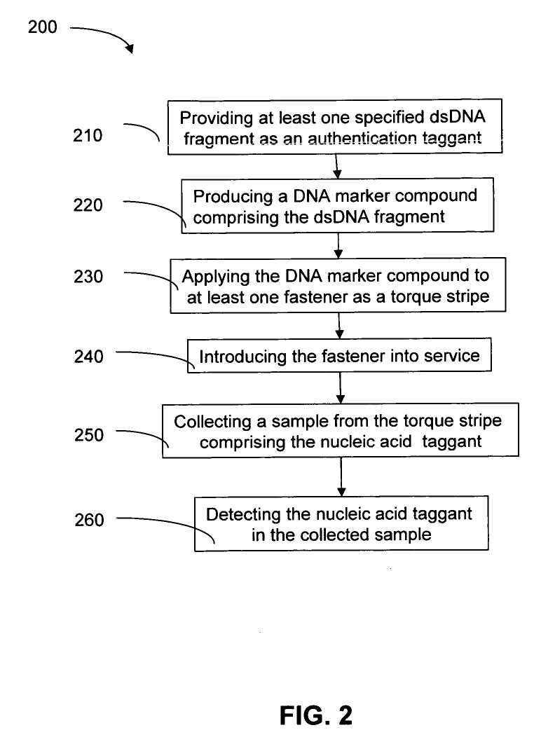

Referring now to FIG. 2, is flow chart of one embodiment of authenticating a particular product with a torque stripe 200 in accordance with the invention. At event 210 a dsDNA taggant produced specifically for a certain fastener manufacturer is provided. The metal fastener comprises a nut and a bolt for an aircraft, and the dsDNA taggant is encapsulated to help prevent degradation of the DNA. A DNA marker compound mixture is providing at event 220 which comprises the encapsulated dsDNA taggant as well as other materials to aid in the longevity of the dsDNA within the marker compound.

The DNA marker compound used to produce a torque stripe on the fastener is in the form of a liquid and is applied to the nut and bolt in event 230. The DNA marker compound may be applied to distinct parts of the nut and bolt to insure the authenticity of individual parts of the fastener or as a torque stripe after the individual parts have been connected to one another correctly. When DNA marker compound placed on a fastener as a torque stripe, there maybe other authentication materials in the marker compound to allow for visible detection of tampering.

In event 240 the fastener is placed into to service on the aircraft or sent through a supply chain by the manufacturer. When the marker compound has been applied as distinct marks on the individual parts of the fastener, the DNA marker helps prevent forgery of the fastener while in the supply chain. When the DNA marker is applied as a torque stripe the fastener can be authenticated during service or maintenance of the aircraft to detect unwanted tampering of the fastener or replacement of inferior fasteners.

At anytime while the fastener is in a supply chain, a sample can be collected from the DNA marker on the fastener to determine the authenticity of the fastener at event 250 of FIG. 2. When the DNA marker is applied as a torque stripe the fastener maybe inspected to determine the authenticity of the fastener during maintenance of the aircraft or during unscheduled inspections of the fastener. Once a sample of the torque stripe has been obtained, the collected sample can be tested for the presence of the DNA marker using techniques such as real time PCR at event 260 after sufficient DNA extraction procedures.

As previously mentioned, the references described in this application are incorporated herein by reference.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The following examples demonstrate the ability to identify a DNA marker from dried torque stripe material. The examples also demonstrate that results could be obtained in a relatively short number of PCR cycles, i.e., within about 30-40 minutes after the start of the PCR reactions.

Target DNA extraction methods were applied to dried torque seal material. DNA was extracted and real-time PCR was used to amplify target DNA (DNA marker) sequences.

Description of PCR Template Primer Sets and Probes

The 872 bp amplicon sequence that was provided by Biowell was analyzed using the LightCycler Probe Design Software 2.0 (version 1.0). The program was used to generate primer and probe sets for real-time PCR. One primer set was constrained so that the forward primer targeted a specific chimeric region of the sequence. A reverse primer and two hybridization probes were designed for use with this primer. Another primer and probe set was created without any constraints. In both cases, the program generated several potential primer and probes combinations. Several of these sets were examined for crosscomplementarity and secondary structure using tools within the LightCycler Probe Design software and also using a variety of other bioinformatic programs. Based on these analyses, the best sets of oligonucleotides were selected.

The primer set and probe selected for the following examples was a nonconstrained set of primers which generated a 123 bp amplicon of the dsDNA marker comprised in the torque seal material. These primers were used in conjunction with a corresponding HybProbe.

Dried Torque Seal Preparation

Five liquid torque seal samples, A-E were obtained and small amounts of the samples were placed on a solid medium in varying amounts for drying the torque seal material. The spots were roughly 0.5 to 0.75 cm in diameter. Several spots were made for each sample, A through E. The spotted torque seal material was allowed to dry undisturbed for varying lengths of time. When wet torque seal material was used, an amount equal to the dried material (˜0.75 cm diameter spot) was placed directly in the microfuge tube with 50 μL water.

DNA Extraction

Grinding Torque Seal Samples

The work surface was cleaned using DNAZap. Dried torque seal samples were placed into a mortar and pestle using forceps. Using a circular grinding motion, the torque seal material was ground for roughly 1 minute, making sure the torque seal pieces were under the pestle for sufficient grinding. After grinding, the torque seal material was placed in a microfuge tube and 50 μL of sterile water was added to the microfuge tube. Vortex The sample was vortexed briefly and centrifuged to bring the solid material to the bottom of the tube. The equipment and work area was cleaned thoroughly between grinding various samples.

The grinding extraction method was carried out in duplicate on torque seal Samples A through E after 7 days or 20 days of drying time.

Samples were stored at 4° C. until further processing (i.e. boiling, PCR purification) or analyzing using primer and HybProbe Sets.

Boiling

The microfuge tubes containing torque seal material (either with or without prior grinding) were boiled in a hot water bath at 95° C. for 10 minutes. After boiling, tubes were placed on ice until PCR reactions were prepared.

QIAquick PCR Purification Kit

In some experiments, an additional method was used after DNA extraction to remove particles that were interfering with the instrument optics. The QIA quick PCR purification kit was used to collect DNA and remove impurities which interfered with the real time PCR signal. In some cases 4 to 6 aliquots of the same torque seal sample (i.e. sample A) were pooled and then purified using the PCR purification kit. The collected DNA was resuspended in 40 μL PCR-grade water.

LightCycler Real-Time PCR Conditions

The real-time PCR reaction solutions were prepared using the LightCycler FastStart DNA MasterPlus kit protocol with 0.5 μM of the forward and reverse primers and 0.15 μM FAM and LC Red probes, followed by adding 0.5 Units/reaction of UNG (Uracil DNA glycosylase) and 2 μL of the torque seal template. Positive, negative and no template controls (NTC) were also included in the experiments. The Roche LightCycler Software (version 4) was used to view amplification curves, crossing points (Cp) and other variables used for data analysis.

Real-time PCR optimization resulted in the use of specific primers and their corresponding hybridization probes. The real-time PCR assay conditions were standard and contained an additional pre-incubation step designed to degrade contaminating amplicon in some experiments. The total time of the PCR run was roughly 1 hour, with results in as little as 30 minutes.

### Example 1

Grinding and Boiling Extraction Method

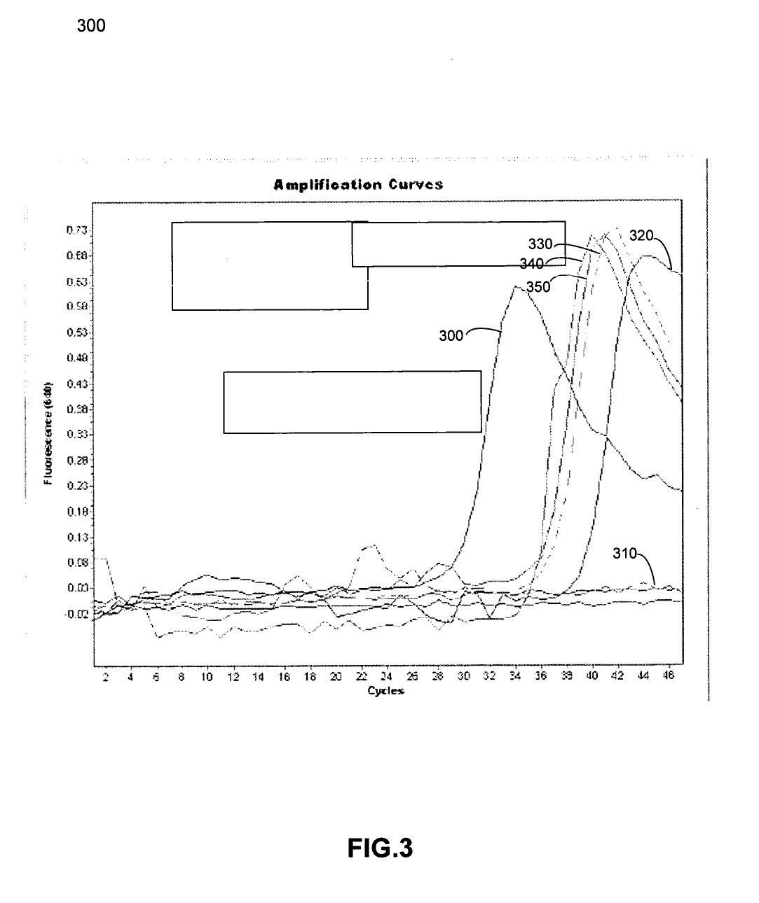

FIG. 3 shows the real time PCR results of torque seal samples which were subjected to the following extraction methods. All of the samples A, B, D, and E were dried for 20 days and then subjected to grinding. Samples D and E were further treated by boiling the grinded sample in water. A positive control 300 for the DNA marker as well as replicate negative controls 310 were also subjected to the real time PCR conditions as the torque seal samples. The positive control 300 has a Cp value of 29 while the negative controls 310 gave only a background signal. The two torque seal samples which were only subjected to grinding, samples A 320 and B 330, gave Cp values of 37 and 33 respectively. The samples which were ground and boiled, samples D 340 and E 350, both had a Cp value of 34. These results demonstrate that the DNA marker can be detected from a sample of torque seal material after exposure for 20 days. Both methods gave qualitative results with the method of grinding followed by boiling giving semi-quantitative results.

### Example 2

Torque Seal Extraction Method with DNA Purification Step

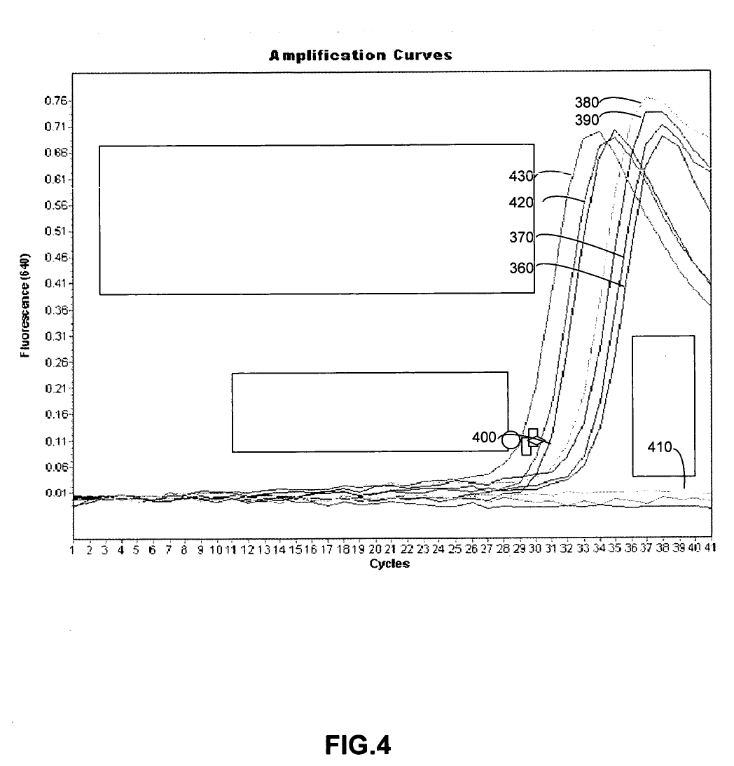

FIG. 4 shows the real time PCR results of torque seal samples which were subjected to the following extraction methods. All of the samples were dried for 20 days, subjected to grinding, dissolved in water and further purified using a QIAgen PCR Purification Kit to remove particulate material that could interfere with the optics of the PCR instrument. The purification step involves centrifugation to bind the DNA present in the torque seal sample to a filter, then wash away any impurities, followed by eluting the DNA with water. The procedure is fast and easy, and takes no more than 15 minutes to perform. Following clean-up, real-time PCR was performed on samples A and B using 2 μL or 4 μL of purified extracted torque seal DNA. We also spiked the pooled samples A and B with 2 μL of positive control DNA (9543-2) to test for inhibitors in the PCR reaction.

The amplification curves of samples A and B after PCR clean-up are shown in FIG. 4. The 2 μL samples of A and B are curves 360 and 370 respectively, and gave similar Cp value of 32. The 4 μL samples of A and B, curves 380 and 390 gave a Cp about 30 and were also fairly reproducible. The positive control 400 amplified as expected with a Cp value of 29 while the replicate negative controls 410 gave no signal above background. Torque samples A and B were spiked with the positive control 420 and 430 respectively, to insure that the purification kit did not effect the amplification of the target DNA. The spiked A and B samples gave Cp values of 27 and 29 respectively. The fluorescent signal generated by samples subjected to the purification kit are smooth and have a typical shape curve for real time PCR. This experiment demonstrates the ability to detect a specific DNA marker from dried torque seal material. The results also indicate that the detection method can be semi-quantitative.

What is claimed is:

1. A method for authenticating a product wherein said product is composed of a plurality of components, said method for authenticating the product comprising:
providing a liquid compound comprising at least one invisible marker, said compound associated with a first entity;
applying said compound to the surface of said product at the juncture of at least two components; and
associating said application of said compound to the at least two components with said first entity.

2. The method of claim 1 further comprising transferring said product to a second entity.

3. The method of claim 2 further comprising inspecting the compound and authenticating that the product is associated with the first entity by detecting said invisible marker.

4. The method of claim 1 wherein said product is integrated into a manufactured product.

5. The method of claim 1 wherein said invisible marker comprises a nucleic acid.

6. The method of claim 1 wherein said invisible marker comprises a UV ink.

7. The method of claim 1 wherein said compound is a paint.

8. A method for authenticating a product wherein said product is composed of a plurality of components, said method for authenticating the product comprising:
providing a paint comprising at least one nucleic acid marker, said compound associated with a first entity;
applying said paint to the surface of said product at the juncture of at least two components; and
associating said application of said paint with said first entity.

9. The method of claim 8 further comprising:
collecting a sample of the paint; and
analyzing the sample for the nucleic acid marker.

10. The method of claim 9 further comprising authenticating that the product is associated with the first entity by detecting the nucleic acid marker.

11. The method of claim 9 wherein the sample is analyzed using PCR.

12. The method of claim 9 further comprising transferring said product along a supply chain.

13. A method for authenticating a particular product, comprising:
providing a marker compound having at least one nucleic acid product applying the marker compound to at least one particular product, which enters at least one supply chain;
collecting a sample of said marker compound from said particular product after said particular product has entered said supply chain; and,
identifying said nucleic acid product in said particular product.

14. The method of claim 13 wherein the particular product comprises at least two parts.

15. The method of claim 14 wherein the marker compound is applied as a torque stripe.

16. The method of claim 15 wherein the nucleic acid product is identified using PCR.

17. The method of claim 14 wherein the particular product is a fastener.
